(19) United States
(12) Patent Application Publication
(10) Pub. No.: US 2012/0172773 A1
(43) Pub. Date: Jul. 5, 2012

(54) DURABLE ELECTRODE CONSTRUCTION FOR AN ORTHOTIC DEVICE

(76) Inventors: David Combs, Oceanside, CA (US); Michael S. Skahan, Escondido, CA (US); Kevin R. Lunau, Valley Center, CA (US)

(21) Appl. No.: 13/395,439

(22) PCT Filed: Sep. 21, 2010
(86) PCT No.: PCT/US2010/049602

§ 371 Date: Mar. 10, 2012

Related U.S. Application Data
(60) Provisional application No. 61/244,824, filed on Sep. 22, 2009.

(51) Int. Cl. A61N 1/36; A61F 5/00

(52) U.S. Cl. 602/2

(57) ABSTRACT

The present invention provides an electrode assembly for attachment to an orthotic device. It includes an electrode carrier. An adhesive layer is removably attached to the electrode carrier. A conductive signal transmission element, e.g. wire, transmits a signal to the electrode assembly. A conductive material layer is attached to the conductive signal transmission element that maintains electrical and mechanical integrity during use within the orthotic device. A conductive pad layer attached to the conductive material layer. One preferred embodiment incorporates a wire including an unexposed portion; and, a conductive attachment means. The electrode assembly preferably includes perforations to facilitate the transmission and evaporation of perspiration or facilitate signal transmission.

### CROSS REFERENCE TO RELATED APPLICATIONS

This application is a non-provisional of and claims priority of U.S. Application Ser. No. 61/244,824 filed Sep. 22, 2009, incorporated herein by reference in its entirety.

### BACKGROUND OF THE INVENTION

1. Field of the Invention

The present invention is directed towards a durable electrode construction, and more particularly, some embodiments of the invention provide a durable electrode construction to be worn with an orthotic device.

2. Description of the Related Art

As discussed here, orthotic devices (orthoses) include any brace, splint, support, or other joint stabilizing means applied to any part of the body to protect, support, or treat biomechanical conditions. Orthotic devices generally include a biomechanical support element that forms the basis of the skeletal or soft tissue support that is required for the majority of these devices.

Orthotic devices must engage effectively with soft tissue in order to provide the desired support. In many parts of the body the soft tissue will move, for example by expanding or contracting as result of muscle or joint movement. For example, the objective of a rigid knee brace is to exert a force on the tibia with respect to the femur in the user's body mass above the knee. By definition, knee braces are applied to soft tissue lying between the brace and the user's skeleton. The rigid element may include some form of liner that contacts the body of the user. The liner may have an outer fabric that is designed to contact the user's skin directly or, alternatively, to engage with clothing that a user may be wearing about the part of the anatomy to which the orthotic device is to be attached. Soft tissue is mobile and in the case of the leg, moves in a cycle corresponding to a user's gait, whether it be through running, walking or other physical movement common to the human knee. The most mobile soft tissue is the quadriceps mechanism lying in front of the femur in the anterior thigh region. The central reference point for a knee brace is the knee joint line. In construction, an orthotic device such as a knee brace would use a joint mechanism, which mimics the movement of the joint to be supported, such as the knee, which is not just a simple hinge. Since each user's body shape is unique, the interface between the orthotic device and the user's leg cannot be predetermined in the manufacture of such a device. This technology can be applied to any brace or support on the body. The knee brace is simply used as an example.

Degenerative joint disease, osteoarthritis, rheumatoid arthritis, repetitive motion, carpal tunnel, tendinitis, and other joint diseases or injuries may be treated through various methods of electrical stimulation. Surface electrical stimulation (SES) treats these conditions using sub-sensory electrical pulses. Other methods of electrostimulation include Transcutaneous Electrical Nerve Stimulation (TENS), Transcutaneous Electrical Stimulation for Arthritis (TESA), Neuromuscular Electrical Stimulation (NMES), Interferential Stimulation (IF), High Volt Galvanic Stimulation, High Volt Pulsed Current (HVPC), Electromagnetic and Pulsed Electromagnetic Field Stimulation, and Micro Current Electrical Stimulation.

When the active user wears an orthotic device with an electrical stimulation assembly attached, there are several potential issues to be solved or minimized. One of these issues is durability, especially in the harsh environment between the device and the user's anatomy. The active user will move, walk, run, jump, and sweat. The assembly's design must be robust to survive this activity. With regards to bodily fluids such as sweat, the electrode assembly attached to the user's anatomy must stay in place, and continue to function well. Current electrodes are very sticky, with no way to ventilate sweat through the assembly.

Electrodes must also peel easily from the anatomy (not accidentally from the electrode carrier) when the orthotic device is removed. When the electrode is ready to be replaced, it must peel relatively easily from its electrode carrier, while not delaminating under normal use. It can be seen that there must be a careful, functional balance in designing the various adhesion layers. With regards to activity levels, current designs include a long wire leading from the signal generator to the electrode worn by the user. This wire can tangle, snag, pull out, and ultimately break electrical connection with the signal generator. The need exists to improve this assembly to a more robust design.

### BRIEF SUMMARY OF EMBODIMENTS OF THE INVENTION

Various embodiments of this invention provide a durable electrode construction to be worn with an orthotic device. Part of this construction can include an electrode carrier to hold the electrode in place. Typical constructions of an electrode carrier may include textile fabric to provide a substrate for the electrode to adhere.

Some embodiments of the invention involve an electrode assembly for attachment to an orthotic device, comprising an adhesive layer with perforations to facilitate the transmission and evaporation of perspiration, an electrical wire for powering the electrode assembly, an adhesive cover for protecting the electrical wire when the adhesive layer is perforated, and a conductive material layer.

As used herein, the term adhesive is defined broadly to include pressure sensitive adhesive, hook and loop fasteners (such as Velcro®), magnets, or other suitable fastening means that have chemical, mechanical, magnetic, or electrostatic adhesion properties.

In certain embodiments, the electrode assembly further comprises a wire holder for supporting the electrical wire, and a conductive protection piece to prevent the electrical wire from physically contacting the conductive material layer. Additionally, the electrode assembly may also include a conductive adhesive to help the electrical wire maintain contact with a conductive protection piece, as well as a conductive adhesive to help the conductive protection piece maintain electrical and mechanical contact with the conductive material layer. In general, the adhesive layer adheres to the conductive layer with a strong bond, and adheres to an electrode carrier with a relatively weaker bond to facilitate the user removing and replacing the electrode assembly. The adhesive layer is configured to leave substantially no residue when peeled from the electrode carrier. The electrode carrier can be designed to fit on or in the orthotic device, and interface with both the user's anatomy and the electrode assembly. In other applications, the electrode carrier itself may be used to hold the electrode to the user's anatomy, without the need for any additional device.

According to some implementations of the invention, the conductive material layer is constructed such that it is resilient to help conform to the user's anatomy. Additionally, the conductive material layer may be constructed to achieve high and consistent electrical conductivity, and is formed to maintain electrical and mechanical integrity. It may be constructed to not break down electrically, mechanically, or otherwise, due to any reaction with the conductive pad layer, adhesive layer, electrical current or bodily fluid. Moreover, the conductive material layer may be constructed to allow the conductive pad layer to adhere well during conditions where the electrode assembly is worn on a user's body. The conductive pad layer also includes a high-tack side that allows it to adhere well to the conductive material layer and a low-tack side that is in contact with a user's anatomy.

In some embodiments, a high tack strength exists between the adhesive layer and the conductive material layer, as well as between the conductive material layer and the conductive pad high-tack side. In such embodiments, a medium tack strength exists between the adhesive layer and an electrode carrier, while a low tack strength exists between the conductive pad low-tack side and the user's skin, thus allowing easy removal of the electrode assembly from either the electrode carrier or the user's body, without delamination.

In some cases, there may exist mechanical bond strength issues with the conductive pad layer (e.g., hydrogel) and the conductive material (e.g., cotton/bamboo/silver fabric). In such cases, bond strength between the conductive material layer and the high-tack side of the conductive pad can be further increased by forcing or “extruding” some of the hydrogel through the conductive material layer fabric. This can be accomplished by rubbing, abrading, or compressing the fabric either during or after the lamination process

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The present invention, in accordance with one or more various embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

Some of the figures included herein illustrate various embodiments of the invention from different viewing angles. Although the accompanying descriptive text may refer to such views as “top,” “bottom” or “side” views, such references are merely descriptive and do not imply or require that the invention be implemented or used in a particular spatial orientation unless explicitly stated otherwise.

FIG. 1 is a plan view of a durable electrode construction to be worn with an orthotic device in accordance with an embodiment of the invention.

FIG. 2 is an exploded view of the durable electrode construction of FIG. 1 in accordance with an embodiment of the invention.

FIG. 3 is a cross sectional view of the durable electrode construction with an electrode carrier incorporated therewith, in accordance with an embodiment of the invention.

FIG. 4 is an exploded view of the durable electrode construction, in accordance with another embodiment of the invention, which utilizes a substantially corrosion-resistant signal transmission means.

FIG. 5 is a cross sectional view of the durable electrode construction of FIG. 4, including conductive adhesive, and substantially corrosion-resistant signal transmission means, in accordance with an embodiment of the invention.

FIG. 6 is an exploded view of the durable electrode construction, including an electrode carrier incorporating a signal transmission means including an unexposed portion; and, a conductive attachment means, in accordance with another embodiment of the invention.

FIG. 7 is a cross sectional view of the durable electrode construction of FIG. 6.

The figures are not intended to be exhaustive or to limit the invention to the precise form disclosed. It should be understood that the invention can be practiced with modification and alteration, and that the invention be limited only by the claims and the equivalents thereof.

### DETAILED DESCRIPTION OF THE EMBODIMENTS OF THE INVENTION

The following diagrams and description present examples of the invention, but in no way, limit the application of the above concepts. The following designs are simply illustrative of their application.

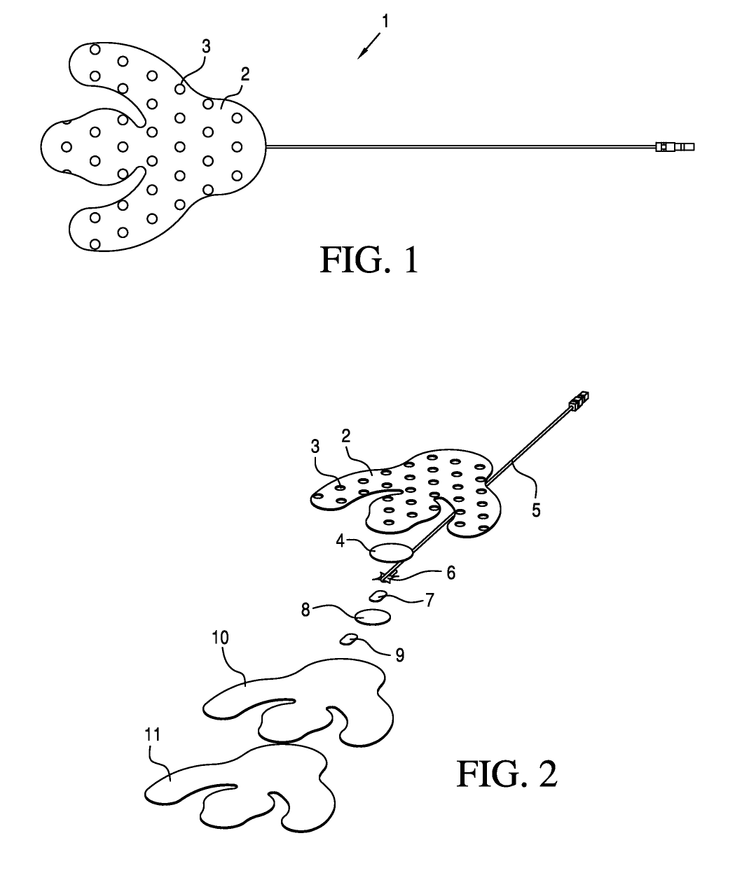

FIG. 1 is a plan view of a durable electrode construction to be worn with an orthotic device in accordance with an embodiment of the invention. In particular, FIG. 1 illustrates an electrode assembly 1 having an adhesive layer 2 with perforations 3 to facilitate the transmission and evaporation of perspiration, for example, from the leg of a wearer of the orthotic device. By way of example, the electrode assembly 1 may be attached to the liner of an orthotic device, for example, a knee brace such as disclosed in U.S. patent application Ser. No. 12/468,794 (US Publication No. 2009/0287126), titled Electrically Stimulating Orthotic Device and Segmented Liner, the contents of which are hereby incorporated herein by reference in its entirety. Other orthotic devices are disclosed in: U.S. patent application Ser. No. 12/782,270, filed May 18, 2010 entitled Bracing and Electrostimulation for Arthritis, is hereby incorporated herein by reference in its entirety. U.S. Pat. No. 7,758,527 (U.S. patent application Ser. No. 10/591,966), entitled Orthotic Device and Segmented Liner, is hereby incorporated herein by reference in its entirety. U.S. patent application Ser. No. 12/510,102 (US Publication No. 2010/0082079), titled Electrodes for Orthotic Device, is hereby incorporated herein by reference in its entirety

As shown in FIG. 1, the electrode assembly 1 may be dimensioned and contoured to fit comfortably around the knee of a user. In further embodiments of the invention, the electrode assembly 1 may include other shapes and contours adapted for use with other areas of the body such as hands, wrists, ankles, shoulders, hips, etc. The electrode assembly 1 is preferably resistant to mechanical shear and the harsh environmental combination of salt and heat. Furthermore, the electrode assembly 1 is preferably resistant to chemical combinations common in electrode construction; for example, electrodes made from hydrogel. By way of example, the electrode assembly 1 may be formed using cotton or bamboo fibers, and featuring silver for conductivity. Alternate materials include, but are not limited to, nylon, polyester, carbon, polyolefin, lycra, stainless steel, etc.

FIG. 2 is an exploded view of the durable electrode construction of FIG. 1 to be worn with an orthotic device in accordance with an embodiment of the invention. As stated, electrode assembly 1 includes an adhesive layer 2 with perforations 3 to facilitate the transmission and evaporation of perspiration. In addition, electrode assembly 1 includes an adhesive cover 4, a signal transmission means (e.g. electrical wire) 5 for powering the electrode assembly 1, a wire holder 6 for supporting electrical wire 5, conductive adhesive 7, a conductive protection piece 8, conductive adhesive 9, a conductive material layer 10, and conductive pad layer 11. The adhesive layer 2 may preferably comprise an acrylic formulation for adhering to the conductive material layer 10. This conductive material layer 10 preferably remains substantially the same conductivity when exposed to the harsh environment and different substances in the hydrogel, and within the orthotic device. Alternate materials for the adhesive layer include, but are not limited to, polyacrylate, rubber-based, silicone, vinyl, etc.

With further reference to FIG. 2, the adhesive cover 4 is used to protect the electrical wire 5 when the adhesive layer 2 is perforated. To avoid wire corrosion, the conductive protection piece 8 is provided to prevent electrical wire 5 from physically contacting the conductive material layer 10 or conductive pad layer 11. Additionally, conductive adhesive 7 is provided to help wire 5 maintain contact with conductive protection piece 8, whereas conductive adhesive 9 is provided to help conductive protection piece 8 maintain contact with conductive material 10 or conductive pad layer 11. During use, the wire holder 6 attaches the electrical wire 5 to conductive protection piece 8, further securing the wire 5.

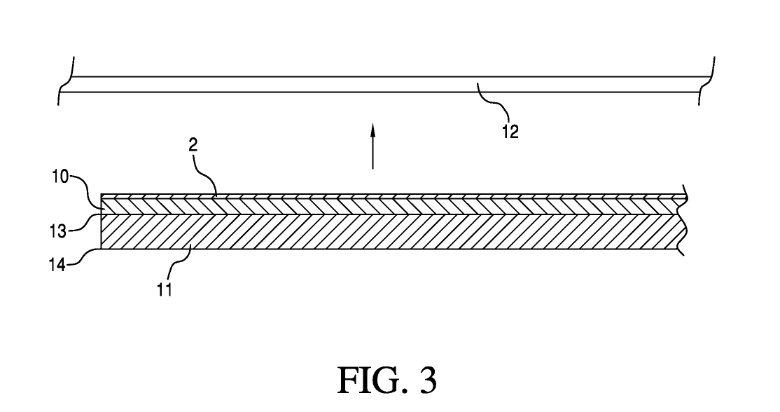

FIG. 3 is a cross sectional view of the durable electrode construction of FIG. 2 in accordance with an embodiment of the invention. The arrow depicts the electrode being placed on an electrode carrier 12. In operation, the adhesive layer 2 is removably attached to the electrode carrier. The adhesive layer 2 must adhere well to the conductive material layer 10, while also adhering well to electrode carrier 12. In particular, the adhesive layer 2 adheres to the conductive layer 10 with a strong bond, yet adheres to the electrode carrier 12 with a weaker bond, to facilitate the user removing and replacing the electrode assembly 1. The adhesive layer 2 preferably leaves substantially no residue when peeled from electrode carrier 12.

With further reference to FIG. 3, the conductive material layer 10 is constructed such that it is resilient to help conform to the user's anatomy. Additionally, the conductive material layer 10 is constructed to achieve high and consistent electrical conductivity (i.e. low electrical impedance). Moreover, the conductive material layer 10 is formed to not break down electrically, mechanically, or otherwise, due to any reaction with the conductive pad layer 11, adhesive layer 2, electrical current, or bodily fluid (such as perspiration, blood, etc.). Electrical breakdown would result in a high (i.e. undesirable) impedance. A preferable impedance range is less than 15,000Ω in a frequency range of 100-1000 Hz. Furthermore, the conductive material layer 10 is constructed to allow the conductive pad layer 11 to adhere well during conditions where the electrode and carrier assembly are worn on the user's body, or between the user's body and an orthotic device. The conductive pad layer 11 has a high-tack side 13 that allows it to adhere well to the conductive material layer 10 and a low-tack side 14 that is in contact with the user's anatomy.

With respect to the above-described electrode assembly 1, the relative adhesive tack strengths or bonds (from highest to lowest) will now be described. These bonds can be quantified by peeling a ¾″ wide strip from its respective substrate. Preferred peel-strength ranges follow each bonding pair below. The highest tack strength exists between adhesive layer 2 and conductive material layer 10, as well as between conductive material layer 10 and conductive pad high-tack side 13, with preferable peel strength ranges of 2-10 lb and 1-10 lb respectively. These adhesive strengths can be different from each other, but are generally higher than the tack strengths listed below. The next highest tack strength exists between adhesive layer 2 and electrode carrier 12, with a preferable peel strength range of 0.3-6 lb. Finally, the lowest tack strength exists between the conductive pad low-tack side 14 and the user's skin, with a preferable peel strength range of 0-4 lb.

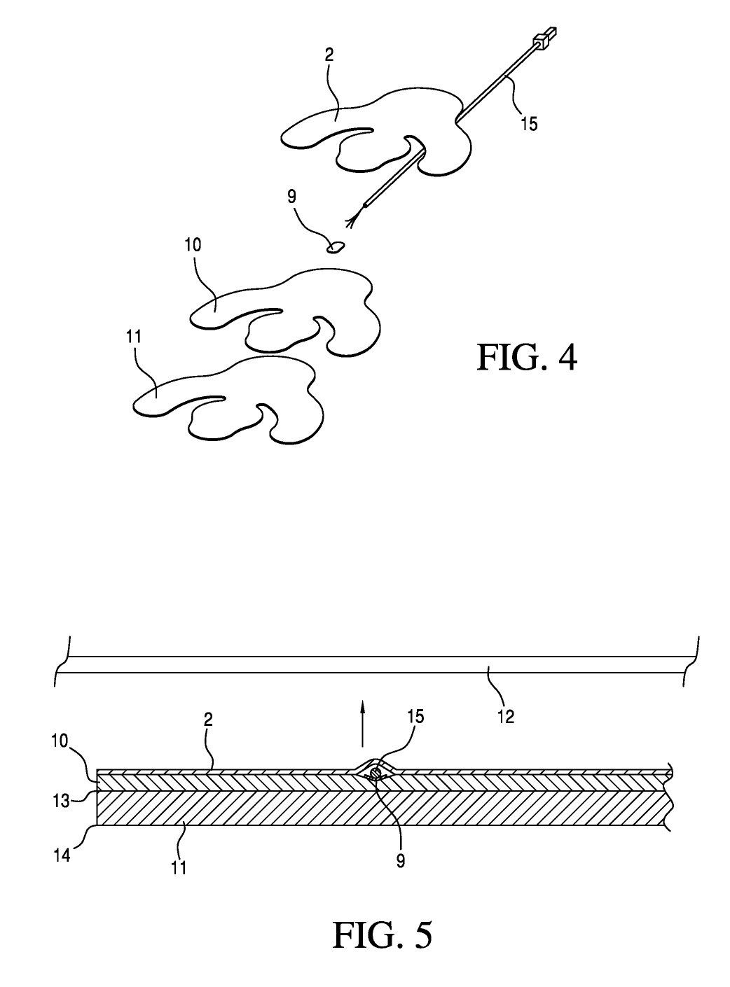

FIG. 4 shows further embodiments of the invention. An alternate electrical wire 5 can be carefully configured to simplify design, construction, and increase conductivity of the electrode assembly 1. Specifically, the alternative electrical wire 5 may obviate the need for the conductive adhesive 7, the conductive protection piece 8, and/or the conductive adhesive 9. To accomplish this, the alternate embodiment of the electrical wire 5 features low impedance comparable to a standard metal wire assembly of similar gage. In addition, the alternative electrical wire 5 is formed to resist corrosion from interaction with the conductive material layer 10, conductive pad layer 11, electrical current, bodily fluids (such as perspiration, blood, etc.), or any combination of the above.

FIG. 4 shows an example of this alternative embodiment using the above-described electrical wire, designated now as 15. Electrical wire 15 can be constructed using a substantially corrosion-resistant material, preferably carbon or a substantially corrosion-resistant metal. This construction preferably omits the corrosion protection elements 4, 7, and 8 in FIG. 2, and leaves only one application of conductive adhesive 9. The embodiment also preferably omits the wire holder 6, since the conductive adhesive 9 or adhesive layer 2 keeps the wire secure in this embodiment. This construction eliminates extra components from the assembly, thus increasing long-term reliability, slims bulk from the design, and increases material resiliency when worn by the active user. Perforation(s) in adhesive layer 2 are not shown, but can be included as described above.

FIG. 5 shows a cross sectional view of the embodiment in FIG. 4. The arrow depicts the electrode being placed on the electrode carrier 12. Electrical wire 15 is placed between adhesive layer 2 and conductive material layer 10 with an application of conductive adhesive 9 to secure.

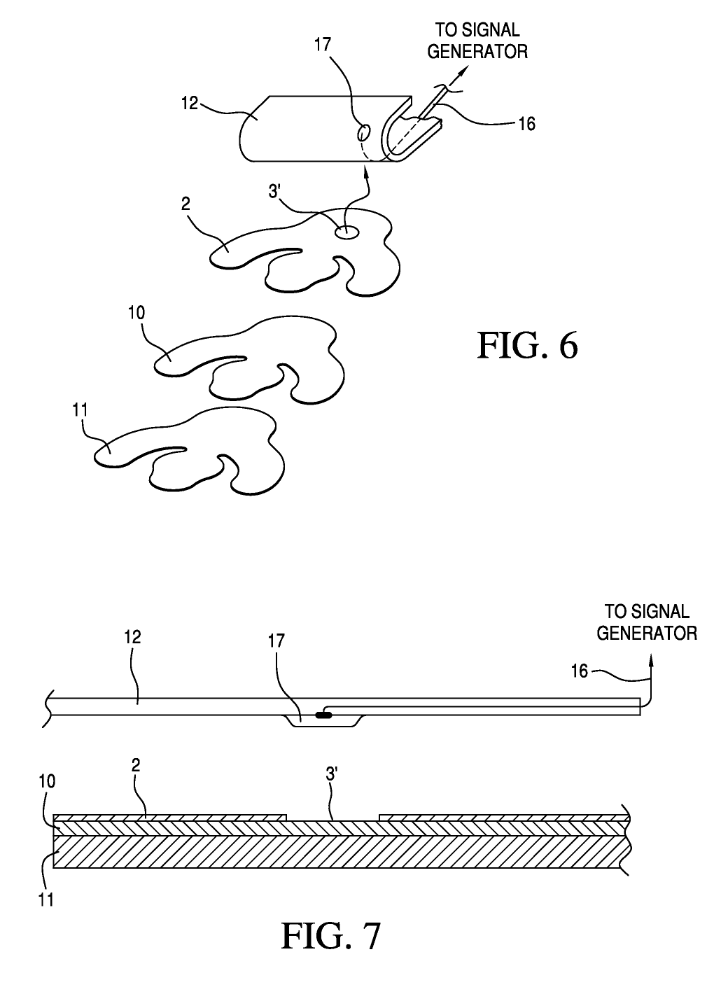

FIG. 6 shows an additional embodiment of the invention, a further alternate embodiment of the electrode assembly 1 omits the wire and associated components altogether, in favor of a conductive attachment signal transmission means 16, 17. In operation, the user simply places the electrode onto the conductive attachment means 17, such that the electrical circuit is complete without the need for a traditional wire. Other embodiments for signal transmission means may include printed or thermally applied conductive material, conductive stitching, or other means to allow integral application of a conductor to transmit the signal. The adhesive layer 2 is constructed such that it exposes conductive attachment means 17 to allow contact with conductive material layer 10. FIG. 6 shows this exposure area 3′ as a perforation, but the exposure means can simply comprise conductive material layer 10 being a different shape than adhesive layer 2, thus allowing exposure to conductive attachment means 17.

The user does not see or interact with the wire 16, since it is embedded within the electrode carrier 12. This minimizes wire pullout or snagging during use since the system is integrated together. By way of example, the conductive attachment means 17 may comprise a conductive hook and loop, a conductive pressure sensitive adhesive, or other mechanical fastening means.

A further embodiment using FIG. 6, includes the adhesive layer 2 itself being electrically conductive. This allows a larger range of positioning on the electrode carrier to connect with the conductive attachment means 17.

FIG. 7 shows a cross section view of the embodiment described above.

While specific embodiments of the invention have been shown in the drawings and described in detail it will be appreciated by those skilled in the art that various modifications and alternatives would be developed in light of the overall teachings of the disclosure. Accordingly, the particular arrangements disclosed herein are meant to be illustrative only and not limiting as to the scope of the invention, which is to be given the full breadth of the appended claims and in any and all equivalents thereof.

While various embodiments of the present invention have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to achieve the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. An electrode assembly for attachment to an orthotic device, comprising:
an electrode carrier;
an adhesive layer removably attached to said electrode carrier;
a conductive signal transmission means for transmitting a signal to the electrode assembly;
a conductive material layer attached to said conductive signal transmission means that maintains electrical and mechanical integrity during use within the orthotic device; and,
a conductive pad layer attached to said conductive material layer.

2. The electrode assembly of claim 1, wherein said conductive signal transmission means is a wire.

3. The electrode assembly of claim 1, wherein said conductive signal transmission means is a carbon wire.

4. The electrode assembly of claim 1, wherein said conductive pad layer includes hydrogel.

5. The electrode assembly of claim 1, wherein said conductive signal transmission means is constructed to maintain substantially the same electrical performance and to not corrode or degrade when used within the orthotic device assembly.

6. The electrode assembly of claim 1, further comprising a wire holder for supporting the conductive signal transmission means.

7. The electrode assembly of claim 1, further comprising a conductive protection piece to prevent the conductive signal transmission means from physically contacting the conductive material layer.

8. The electrode assembly of claim 1, further comprising a conductive adhesive to help the conductive signal transmission means maintain contact with a conductive protection piece.

9. The electrode assembly of claim 1, further comprising a conductive adhesive to help the conductive protection piece maintain contact with the conductive material layer.

10. The electrode assembly of claim 1, further comprising a resilient conductive adhesive to facilitate fit on the user's anatomy.

11. The electrode assembly of claim 1, wherein said adhesive layer adheres to the conductive layer with a strong bond, and adheres to said electrode carrier with a relatively weaker bond to facilitate the user removing and replacing the electrode assembly.

12. The electrode assembly of claim 1, wherein said adhesive layer is configured to leave substantially no residue when peeled from said electrode carrier.

13. The electrode assembly of claim 1, wherein the conductive material layer is formed from one or more materials selected from the group consisting of: cotton, bamboo, nylon, polyester, polyolefin, lycra, stainless steel, carbon, and silver.

14. The electrode assembly of claim 1, wherein said conductive material layer is constructed such that it is resilient to help conform to the user's anatomy.

15. The electrode assembly of claim 1, wherein said conductive material layer is constructed to achieve low electrical impedance.

16. An electrode assembly for attachment to an orthotic device, comprising:
an electrode carrier;
an adhesive layer removably attached to said electrode carrier;
a conductive signal transmission means for transmitting a signal to the electrode assembly;
a conductive material layer attached to said conductive signal transmission means that is formed to not break down electrically, or mechanically during use within the orthotic device; and,
a conductive pad layer attached to said conductive material layer.

17. The electrode assembly of claim 16, wherein said conductive material layer is constructed to allow the conductive pad layer to adhere well during conditions where the electrode assembly is worn on a user's body, or between a user's body and an orthotic.

18. The electrode assembly of claim 16, wherein said conductive signal transmission means is constructed at least partially within said electrode carrier.

19. The electrode assembly of claim 16, wherein said adhesive layer comprises at least one perforation to facilitate the transmission and evaporation of perspiration.

20. The electrode assembly of claim 16, wherein said conductive pad layer includes a high-tack side that allows it to adhere well to the conductive material layer and a low-tack side that is in contact with a user's anatomy.

21. The electrode assembly of claim 20, wherein a relatively high-tack strength exists between said adhesive layer and said conductive material layer, as well as between said conductive material layer and said conductive pad high-tack side.

22. The electrode assembly of claim 20, wherein a relatively medium tack strength exists between said adhesive layer and said electrode carrier.

23. The electrode assembly of claim 20, wherein a relatively low tack strength exists between said conductive pad low-tack side and the user's skin.

24. The electrode assembly of claim 20, wherein said conductive pad layer includes hydrogel.

25. The electrode assembly of claim 24, wherein the bond strength between the conductive material layer and the high-tack side of the conductive pad is further increased by forcing or extruding some of said hydrogel through said conductive material layer.

26. An electrode assembly for attachment to an orthotic device, comprising:
an electrode carrier;
at least one conductive signal transmission means for transmitting a signal to the electrode assembly;
at least one conductive attachment means that connects to said signal transmission means and to said electrode carrier;
an adhesive layer for removable attachment to said electrode carrier;
a conductive material layer which is exposed in at least one location to said conductive attachment means, said at least one conductive attachment means for carrying said signal from said signal transmission means to said conductive material layer; and,
a conductive pad layer attached to said conductive material layer for transmitting a signal to user's anatomy.

27. The electrode assembly of claim 26, wherein said conductive signal transmission means is constructed at least partially integral with said electrode carrier.

28. An electrode assembly for attachment to an orthotic device, comprising:
an electrode carrier;
at least one conductive signal transmission means for transmitting a signal to the electrode assembly;
at least one conductive attachment means that connects to said signal transmission means and to said electrode carrier;
an adhesive layer for removable attachment to said electrode carrier, said adhesive layer being electrically conductive for transmitting said signal from said conductive attachment means to said conductive pad layer;
a conductive material layer, said at least one conductive attachment means for carrying said signal from said signal transmission means to said conductive material layer; and,
a conductive pad layer attached to said conductive material layer for transmitting a signal to user's anatomy.
